(19) United States
(12) Patent Application Publication
(10) Pub. No.: US 2007/0072845 A1
(43) Pub. Date: Mar. 29, 2007

(54) Process for the preparation of olanzapine form 1 useful as antipsychotic drug

(76) Inventors: Davuluri Rammohan Rao, Hyderabad (IN); Shriprakash Dhar Dwivedi, Hyderabd (IN); Pamujula Sreenivasulu, Hyderabad (IN); Surapaneni Sasi Kiran, Hyderabad (IN)

Correspondence Address: MOORE & VAN ALLEN PLLC, P.O. BOX 13706, Research Triangle Park, NC, 27709, US

(21) Appl. No.: 10/557,650

(22) PCT Filed: Jul. 16, 2004
(86) PCT No.: PCT/IN04/00210

§ 371 Date: Nov. 18, 2005

(30) Foreign Application Priority Data
Feb. 19, 2004 (IN) 128 CHE 2004

(51) Int. Cl. A61K 31/551; C07D 498/02

(52) U.S. Cl. 514/220; 540/560

(57) ABSTRACT

This invention provides an improved process for the preparation of Olanzapine Form (I). Olanzapine, which is 2-methyl-4-(4-methyl-1-piperazinyl)-10H-thieno[2,3-b][1,5]benzodiazapine. More specially, the invention provides insitu improved process for the direct preparation of crystalline form of Olanzapine Form (I). The present invention also provides high pure Olanzapine Form I with single individual impurity less than 0.1% by HPLC.

### FIELD OF THE INVENTION

This invention provides an improved process for the preparation of Olanzapine Form I. Olanzapine Form I, which is 2-methyl-4-(4-methyl-1-piperazinyl)-10H-thieno[2,3-b][1,5]benzodiazapine Form-I. Olanzapine Form I, has the formula I given below. More specially, the invention provides an improved process for the direct preparation of crystalline form of Olanzapine Form-I.

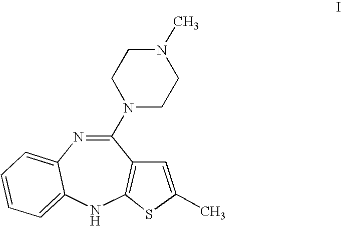

Olanzapine Form I of the Formula I is a pharmaceutical compound useful as a typical antipsychotic drug.

### BACK GROUND OF THE INVENTION/PRIOR ART

The Olanzapine was first disclosed in U.S. Pat. No. 5,229,382. The process disclosed in the said patent includes, condensing propanaldehyde with malanonitrile and sulphur in the presence of N,N-Dimethylformamide and triethylamine to give 2-amino-5-methyl thiophene-3-carbonitrile, which on condensation with 2-fluoronitro benzene gave 2-(2-nitro anilino)-5-methyl thiophene-3-carbonitrile, which on reduction and cyclization with stannous chloride gave 4-Amino-2-methyl-10H-thieno[2,3-b][1,5]benzodiazepine hydrochloride. The 4-Amino-2-methyl-10H-thieno[2,3-b][1,5]benzodiazepine hydrochloride salt was condensed with N-Methylpiperazine in toluene and dimethyl sulphoxide to give Olanzapine which on crystallization in Acetonitrile to give Olanzapine (Pure).

The crystal form of Olanzapine has disclosed in the U.S. Pat. No. 5,736,541. As described in U.S. Pat. No. 5,736,541, the synthesis of Olanzapine according to the methods described in U.S. Pat. No. 5,229,382 produces a metastable, dull colored product referred to in the 5, 736, 541 Patent as Form-I and not well suited for commercial use in pharmaceutical formulations.

The U.S. Pat. No. 5,736,541 discloses and claims a more stable polymorphic form of Olanzapine, designated as Form-II, a method to produce Form-II Olanzapine and pharmaceutical compositions containing Form-II.

Olanzapine. Form-I and Form-II are characterized in the U.S. Pat. No. 5,736,541 by powder X-ray diffraction. The inter planar spacings (d-spacings) and typical relative intensities (I/I₁) are reported.

As per the U.S. Pat. No. 5,736,541, a typical example of an X-ray diffraction pattern for Olanzapine Form I and Form II is as follows wherein d represents the inter planar spacing and I/I₁ reports the typical relative intensities.

| | |
|---|---|
| Olanzapine Form-I | Olanzapine Form-II |

| d | I/I₁ | d | I/I₁ |
|---|---|---|---|
| 9.9463 | 100.00 | 10.2689 | 100.00 |
| 8.5579 | 15.18 | 8.577 | 7.96 |
| 8.2445 | 1.96 | 7.4721 | 1.41 |
| 6.8862 | 14.73 | 7.125 | 6.50 |
| 6.3787 | 4.25 | 6.1459 | 3.12 |
| 6.2439 | 5.21 | 6.071 | 5.12 |
| 5.5895 | 1.10 | 5.4849 | 0.52 |
| 5.3055 | 0.95 | 5.2181 | 6.86 |
| 4.9815 | 6.14 | 5.1251 | 2.47 |
| 4.8333 | 68.37 | 4.9874 | 7.41 |
| 4.7255 | 21.88 | 4.7665 | 4.03 |
| 4.6286 | 3.82 | 4.7158 | 6.80 |
| 4.533 | 17.83 | 4.4787 | 14.72 |
| 4.4624 | 5.02 | 4.3307 | 1.48 |
| 4.2915 | 9.19 | 4.2294 | 23.19 |
| 4.2346 | 18.88 | 4.141 | 11.28 |
| 4.0855 | 17.29 | 3.9873 | 9.01 |
| 3.8254 | 6.49 | 3.7206 | 14.04 |
| 3.7489 | 10.64 | 3.5645 | 2.27 |
| 3.6983 | 14.65 | 3.5366 | 4.85 |
| 3.5817 | 3.04 | 3.3828 | 3.47 |
| 3.5064 | 9.23 | 3.2516 | 1.25 |
| 3.3392 | 4.67 | 3.134 | 0.81 |
| 3.2806 | 1.96 | 3.0848 | 0.45 |
| 3.2138 | 2.52 | 3.0638 | 1.34 |
| 3.1118 | 4.81 | 3.0111 | 3.51 |
| 3.0507 | 1.96 | 2.8739 | 0.79 |
| 2.948 | 2.40 | 2.8102 | 1.47 |
| 2.8172 | 2.89 | 2.7217 | 0.20 |
| 2.7589 | 2.27 | 2.6432 | 1.26 |
| 2.6597 | 1.86 | 2.6007 | 0.77 |
| 2.6336 | 1.10 | | |
| 2.5956 | 1.73 | | |

U.S. Pat. No. 5,703,232 claims lower alcohol solvates of Olanzapine referred in this patent as Form-I and methods for their preparation. The polymorph designated as Form I in this patent has the same characteristic inter planar spacing by X-ray diffraction as Form II disclosed in the above mentioned U.S. Pat. No. 5,736,541 and should therefore be considered as Form II. In the U.S. Pat. No. 5,703,232 the Form prepared has the same characteristic inter planar spacing by X-ray diffraction as the polymorph designated as Form-II in the U.S. Pat. No. 5,736,541 and therefore should thus be considered same polymorph.

U.S. Pat. No. 5,637,584 provides a methylene chloride crystalline solvate of 2-methyl-4-(4-methyl-1-piperazinyl)-10H-thieno[2,3-b][1,5]benzodiazepine (Olanzapine) and also provides a process for preparing anhydrous Form I using a methylene chloride solvate comprising drying such methylene chloride solvate and crystallizing the dried material in a solvate selected from the group consisting of aromatic hydrocarbons, C₃-C₉ ketones, C₃-C₉ branched alcohols, C₃-C₉ esters, C₃-C₉ hydrocarbons, C₃-C₉ ethers and cyclic ethers in the presence of Form I 2-methyl-4-(4-methyl-1-piperazinyl)-10H-thieno [2,3-b][1,5]benzodiazepine.

U.S. Pat. No. 3,631,250 discloses a compound selected from the group consisting of a methanol, ethanol, 1-propanol crystalline solvates of 2-methyl-4(4-methyl-1-piperazinyl)-10H-thieno[2,3-b][1,5]benzodiazepine (Olanzapine) and provides a process for preparing anhydrous Form I comprising contesting a lower alcohol solvate with a solvent selected from the group consisting of ethyl acetate, acetone, 2-propanol, t-butanol, tetrahydrofuran, and toluene and also provides a new method for preparing a lower alcohol solvate of Olanzapine.

WO Patent WO 02/18390A1 describes a method for the preparation of hydrates of Olanzapine and its conversion into a pure crystalline form of Olanzapine Form-I and also describes a method of converting Olanzapine Form-II to From-I using methylene chloride as a solvent.

### OBJECTIVE OF THE PRESENT INVENTION

The main objective of the present invention is to provide an improved, direct and simple process for the preparation of Olanzapine Form I, which is a typical antipsychotic drug overcoming the disadvantages of the prior art.

Another objective of the present invention is to provide an improved process for the preparation of Olanzapine Form I by insitu process.

According to the present invention, there is provided an improved process for the preparation of Olanzapine Form I which comprises:
i) Refluxing a mixture of 4-Amino-2-methyl-1H-thieno[2,3-b][1,5]benzo diazepine hydrochloride, N-methyl piperazine, dimethyl sulphoxide and toluene under inert atmosphere for a period in the range of 5-25 hours at a temperature in the range of 110-130° C., preferably in the range of 122-125° C.
ii) Cooling the resulting reaction mixture to a temperature in the range of 20-90° C. iii) Adding demineralized water (DM water) to the cooled mixture.
iv) Cooling the resulting mixture to a temperature in the range of −10 to 30° C. preferably 0-5° C. and stirring for a period in the range of 2-10 hours.
v) Filtering the mixture and sucking dry for a period in the range of 0.5-2 hours.
vi) Slurring the resulting wet cake with DM water at a temperature in the range of 50-90° C. for a period in the range of 20 min to 3 hours.
vii) Filtering the material and sucking dry for a period in the range of 0.5 to 2 hours.
viii) Repeating the steps (vi) to (vii) till the traces of dimethyl sulphoxide and its odour are removed
ix) Dissolving the resulting wet cake in a chlorinated solvent preferably methylene chloride at a temperature in the range of 25-30° C.
x) Separating the aqueous layer, if any.
xi) Stirring the organic layer i.e. methylene chloride layer with anhydrous sodium sulfate or anhydrous magnesium sulfate for a period in the range of 20 min. to 2 hours.
xii) Filtering and washing with methylene chloride.
xiii) Repeating the steps (xi) & (xii) till the moisture content is less than 0.1% drying the methylene chloride layer repeatedly with anhydrous magnesium sulfate upto the moisture content below 0.1% at 25-30° C. is necessary.
xiv) Purging dry ammonia gas in methylene chloride layer of step (xiii) upto saturation at a temperature in the range of 25-30° C. in the presence of the anhydrous magnesium sulfate for getting the consistent polymorphic form of Olanzapine Form I.
xv) Removing the magnesium sulphate salts from the reaction mixture and washing the salts with methylene chloride.
xvi) Refluxing the methylene chloride layer for a period in the range of 30 min to 2 hours.
xvii) Concentrating the reaction mixture under vacuum (600-650 mm/Hg) upto product isolation takes place and distilling the methylene chloride under vacuum
xviii) Cooling the reaction mixture to a temperature in the range of −10 to 25° C. preferably 0-2° C.
xix) Stirring the material for a period in the range of 30 min. to 5 hours preferably 1-2 hours at a temperature in the range of 0-5° C.
xx) Filtering the material and washing with chilled methylene chloride
xxi) Air drying the material at a temperature in the range of 25-30° C. for a period in the range of 10-15 hours and
xxii) Vacuum drying the product with continuously maintaining a vacuum (600-650 mm/Hg) at a temperature in the range of 60-70° C. for a period in the range of 12-72 hours without breaking the vacuum to avoid the impurity formation.

In a preferred embodiment of the invention, in the step (i), the refluxing may be effected preferably by 15-20 hours and at a temperature preferably in the range of 110-130° C., more preferably in the range of 122-125° C.

In the step (vi) the stirring may be done preferably at a temperature in the range of 60-65° C. preferably for a period in the range of 20 min to 3 hours, more preferably for 45 min. to 2 hours.

The traces of dimethyl sulphoxide and its odour may be removed in step (viii) by repeating the slurry of wet cake in water at 50-90° C. for 3 to 4 times is necessary. If the dimethyl sulfoxide odour is not removed totally, it will lead to failure in the formation of Olanzapine Form I.

The moisture of Methylene chloride layer may be removed in step No (xiii) by repeating Magnesium sulphate treatment, till moisture content of methylene chloride layer is below 0.1%. If moisture content of methylene chloride layer is more than 0.1%, it results the failure of the formation of Olanzapine Form I.

Distillation of Methylene chloride under vacuum in step No. (xvii) also have a key role for impurity profile while preparation of Olanzapine Form I. Impurity formation is observed when the methylene chloride layer is distilled atmospherically at 45-50° C., whereas methylene chloride is distilled under vacuum (600-650 mm.Hg) below 30° C. gave HPLC purity 99.92% with single individual impurity less than 0.1%

The details of the invention are given in the Examples given below which are provided solely to illustrate the present invention, therefore they should not be construed to limit the scope of the invention. Many other specific embodiments of the present invention which will be obvious and apparent to one skilled in the art from the foregoing disclosure are also fall with in the scope of the present invention.

### EXAMPLE 1

A mixture of 4-Amino-2-methyl-10H-thieno-[2,3-b][1,5]benzodiazepine hydrochloride (100 g), N-Methyl piperazine (312.76 g), dimethyl sulfoxide (460 ml) and toluene (460 ml) were heated to reflux. The reaction mixture was maintained at reflux for 20 hours. Completion of the reaction was monitored by Thin layer chromatography (TLC) and then cooled to 40-50° C. Water (460 ml) was added to the reaction mixture and further cooled to 0-5° C. The reaction mixture was maintained at 0-5° C. for 2 hours and filtered. Yield: 127 g (wet). Water (600 ml) & wet material were stirred at 60-65° C. for 45 min. and filtered. This was repeated three to four times to remove the traces of dimethyl sulfoxide and its odour. Yield: 119 g (wet). Wet material was dissolved in methylene chloride (3000 ml) and water was separated. The methylene chloride (organic) layer was dried with anhydrous magnesium sulfate repeatedly (100 g each time) upto moisture content of organic layer was below 0.1%. Dry ammonia gas was purged into the organic layer while stirring with anhydrous magnesium sulfate (50 g) at 25-30° C. upto saturation. The magnesium sulfate salts were filtered and washed with methylene chloride (100 ml). The organic layers were mixed and refluxed for 30 min. The organic layer was concentrated under vacuum (600-650 mm/Hg) upto product isolation was started. The reaction mixture was cooled to 0-5° C. and maintained for 1 hr. at 0-5° C. Product was filtered and washed with chilled methylene chloride (50 ml) Yield: 93.0 g. The product was air dried for 12 hours. Yield: 72.0 g. The air dried product was further dried under vacuum (600-650 mm/Hg) at 60-70° C. for 48 hours continuously gave 99.92% pure Olanzapine Form 1. Yield: 61.5 g. The inter planer spacing (d-spacings) and typical relative intensities (I/I₁) of powder X-ray diffraction were as follows.

| d-value | I/I₁ |
|---|---|
| 9.92608 | 100.0 |
| 8.52807 | 29.5 |
| 8.19986 | 18.2 |
| 6.87492 | 12.9 |
| 6.36322 | 5.6 |
| 5.91157 | 4.5 |
| 5.58243 | 3.0 |
| 4.97055 | 7.2 |
| 4.82631 | 80.0 |
| 4.73300 | 32.2 |
| 4.61640 | 24.8 |
| 4.52781 | 34.8 |
| 4.23463 | 19.4 |
| 4.08680 | 34.0 |
| 3.82153 | 8.5 |
| 3.75505 | 21.1 |
| 3.69123 | 40.0 |
| 3.57709 | 7.1 |
| 3.50332 | 9.4 |
| 3.34467 | 7.8 |
| 3.23650 | 6.1 |
| 3.10616 | 7.6 |
| 3.03905 | 3.5 |
| 2.88339 | 2.6 |
| 2.82043 | 3.9 |
| 2.75676 | 5.8 |
| 2.59189 | 5.8 |
| 2.46045 | 6.6 |
| 2.38299 | 4.3 |
| 2.32966 | 5.5 |
| 2.12544 | 3.9 |
| 2.05751 | 2.6 |

### EXAMPLE 2

A mixture of 4-Amino-2-methyl-10H-thieno-[2,3-b][1,5]benzodiazepine hydrochloride (25 g), N-Methyl piperazine (78.19 g), dimethyl sulfoxide (115 ml) and toluene. (115 ml) were heated to reflux. The reaction mixture was maintained at reflux for 20 hours completion of the reaction was monitored by Thin layer chromatography (TLC) and then cooled to 40-50° C. Water (115 ml) was added to the reaction mixture and further cooled to 0-5° C. The reaction mixture was maintained at 0-5° C. for 2 hours and filtered. Yield: 32.0 g (wet). Wet material was charged 150 ml water and stirred at 60-65° C. for 45 min. followed by filtration. Wet material and water (150 ml) were stirred at 60-65° C. and repeated three to four times to remove the traces of dimethyl sulfoxide and its odour. Yield: 31.3 g (wet). Wet material was dissolved in chloroform (750 ml) and water was separated. The chloroform (organic) layer was dried over anhydrous magnesium sulfate repeatedly (25 g each time) till the moisture content of organic layer was below 0.1%. Dry ammonia gas was purged into the organic layer while stirring with anhydrous magnesium sulfate (12.5 g) at 25-30° C. upto saturation. The magnesium sulfate salts were filtered and washed with chloroform (25 ml). The organic layers were combined and refluxed for 30 min. The organic layer was concentrated under vacuum (600-650 mm/Hg) upto product isolation was started. The reaction mixture was cooled to 0-5° C. and maintained for 1 hour at 0-5° C. Product is filtered and washed with chilled chloroform (12.5 ml) Yield: 11.6 g (wet). The product was air dried for 12 hours. Yield: 10.0 g. The air dried product was further dried under vacuum (600-650 mm/Hg) at 60-70° C. for 48 hours continuously gave 99.50% pure Olanzapine Form I. Yield: 8.5 g. The inter planer spacing (d-spacings) and typical relative intensities (I/I₁) of powder X-ray diffraction were as follows.

| d-value | I/I₁ |
|---|---|
| 9.84970 | 100.0 |
| 8.47167 | 6.8 |
| 8.14210 | 7.1 |
| 6.82006 | 4.3 |
| 6.32759 | 2.3 |
| 5.54555 | 1.7 |
| 4.95573 | 3.4 |
| 4.79761 | 25.7 |
| 4.71634 | 17.3 |
| 4.59634 | 10.1 |
| 4.50564 | 12.1 |
| 4.22111 | 9.8 |
| 4.07202 | 12.2 |
| 3.74199 | 8.2 |
| 3.68616 | 12.2 |
| 3.55808 | 2.0 |
| 3.49212 | 2.7 |
| 3.23037 | 2.6 |
| 3.09860 | 3.0 |
| 2.80907 | 2.0 |
| 2.74570 | 1.6 |
| 2.58950 | 1.8 |
| 2.45437 | 1.9 |
| 2.32630 | 2.0 |

The aforementioned crystalline forms prepared by the process described in the Examples 1 to 2 have been examined for their structural and analytical data viz., powder X-ray diffraction (XRD) and Infrared absorption spectroscopy (IR). The results obtained are discussed and the respective drawings attached (FIG. 14).

The X-ray diffraction pattern setout herein for examples 1 to 2 were obtained using X-ray diffractrometer having a copper anode, radiation source of wave length λ=1.5406A°. The samples were scanned between 2.000° to 50.000° of 2-theta scale.

In the drawings

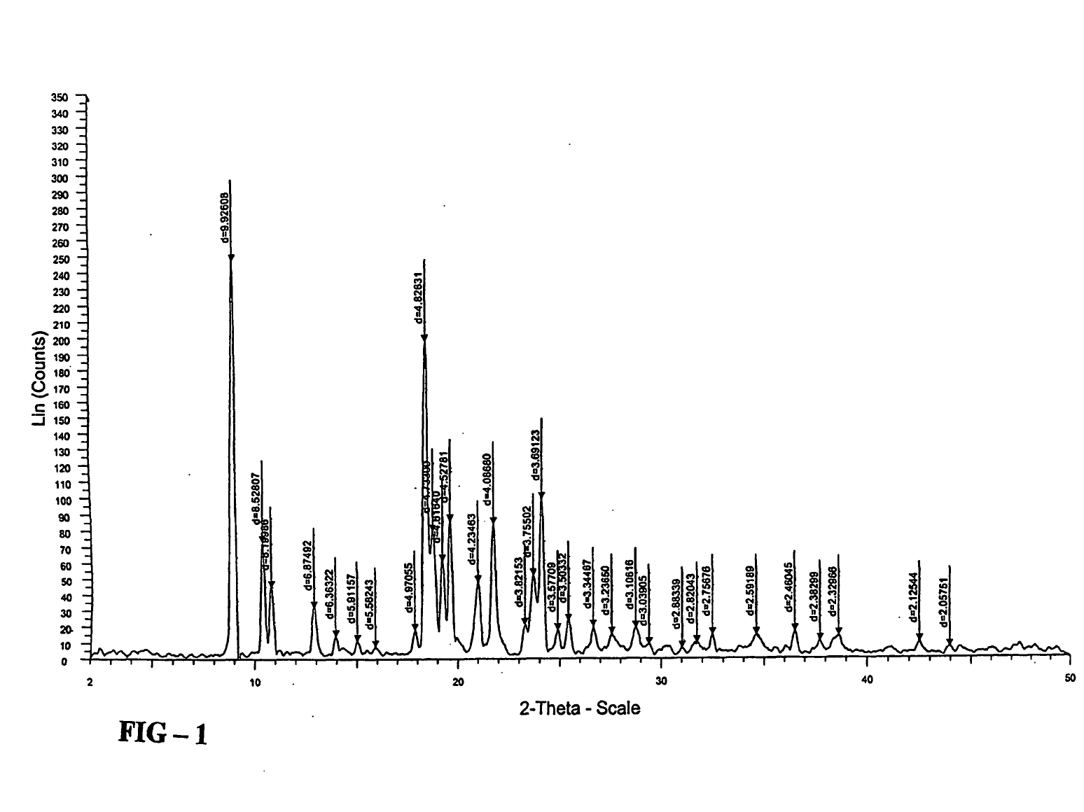

FIG. 1 is a characteristic X-ray powder diffraction pattern of Olanzapine Form I obtained by the process described in the Example—1 (vertical axis: Lin (counts); Horizontal axis: 2-theta scale).

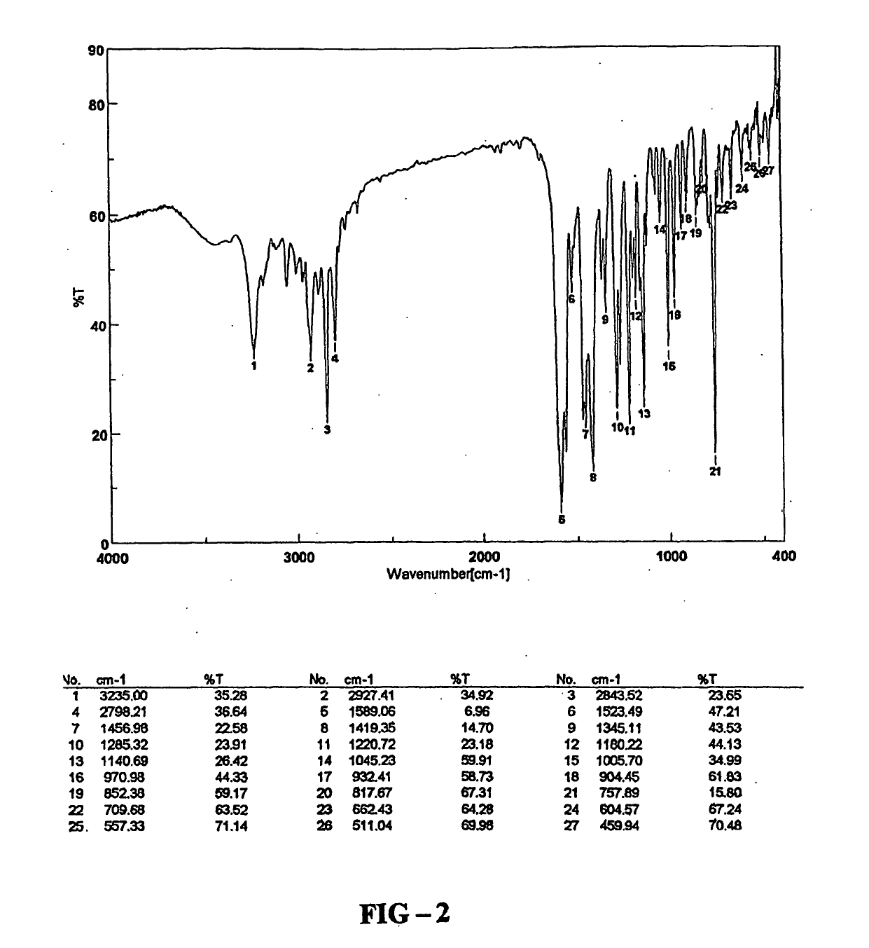

FIG. 2 is a characteristic infrared as absorption spectrum in potassium bromide of Olanzapine Form I obtained by the process described in the Example—1 (vertical axis, tramission (%); Horizontal axis: wave number (cm⁻¹)).

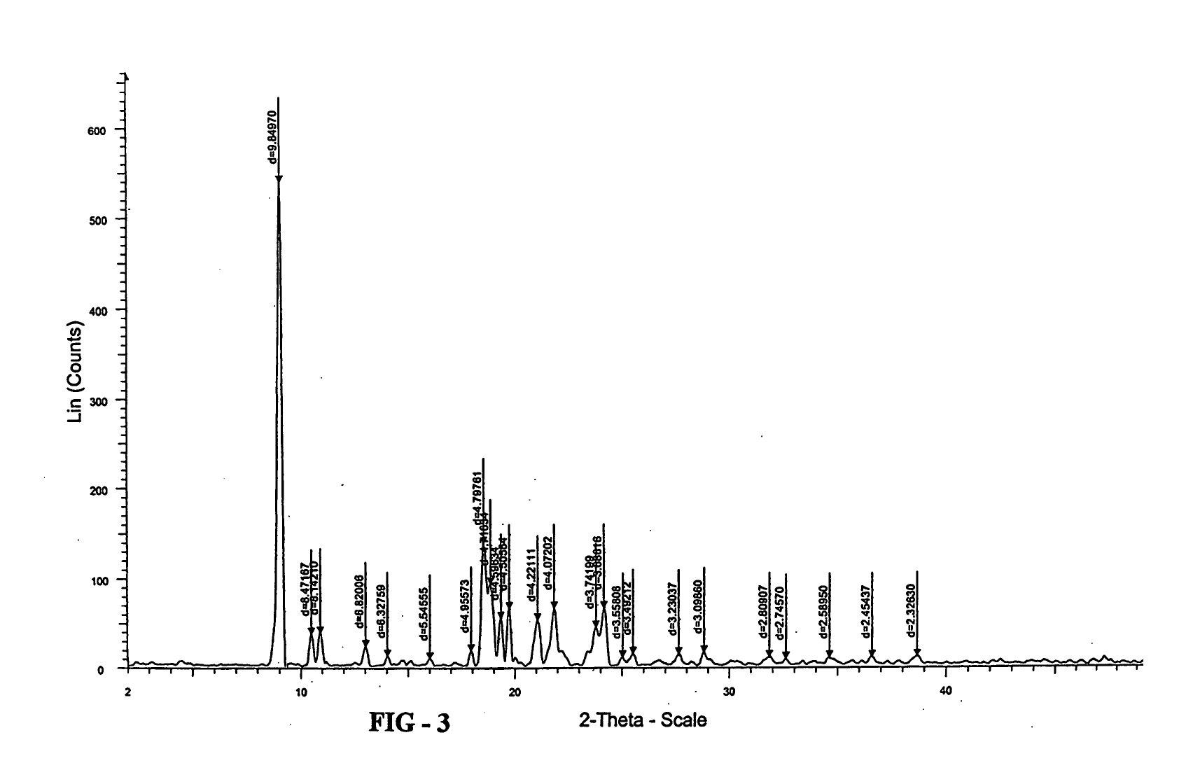

FIG. 3 is a characteristic X-ray powder diffraction pattern of Olanzapine obtained by the process described in the Example—2 (vertical axis: Lin (counts); Horizontal axis 2-theta scale).

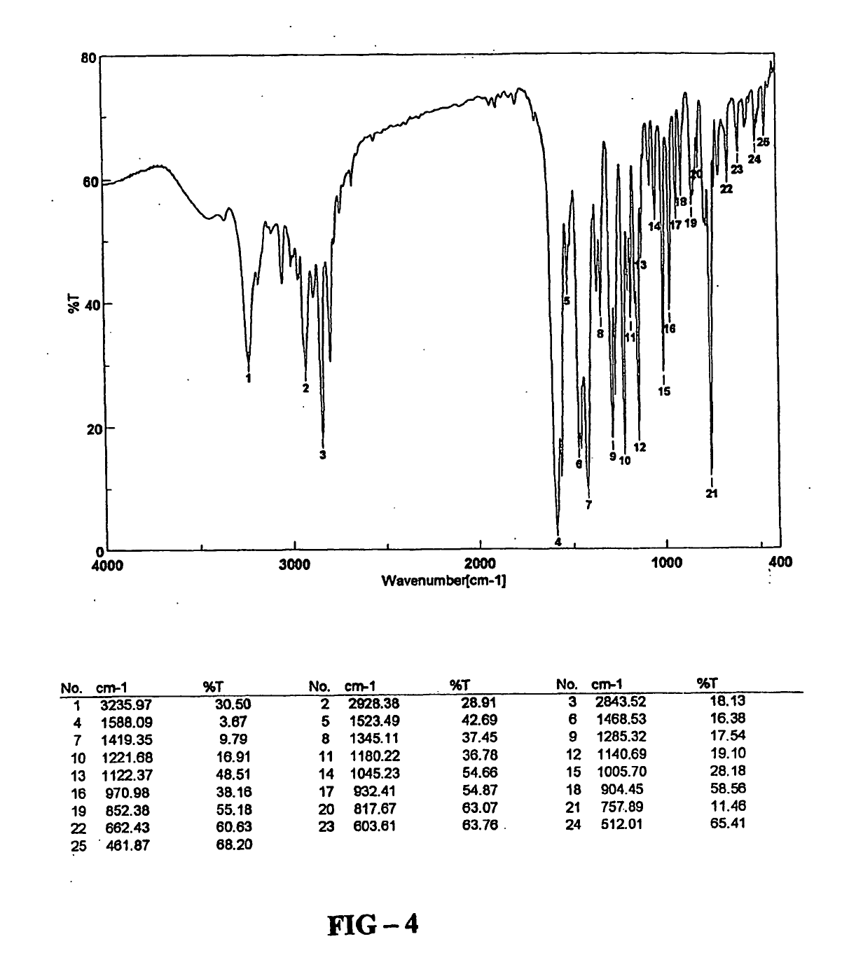

FIG. 4 is a characteristic infrared as absorption spectrum in potassium bromide of Olanzapine obtained by the process described in the Example—2. (vertical axis, tramission (%); Horizontal axis: wave number (cm⁻¹)).

### ADVANTAGES OF THE INVENTION

1. The present invention is simple and insitu process for direct preparation of Olanzapine Form I.
2. The other advantage of the present invention is to get consistently polymorphic form I by controlling following parameters.
   i) Removal of dimethyl sulphoxide and its odour by repeating the slurry of wet cake in hot water.
   ii) Drying the methylene chloride layer repeatedly with anhydrous magnesium sulphate upto the moisture content below 0.1% at 25-30° C.
   iii) Purging dry ammonia gas in methylene chloride upto saturation at 25-30° C. in the presence of the anhydrous magnesium sulphate.
3. The present process results in getting polymorphic Form I with single individual impurity less than 0.1%.
4. The process is simple, safe and can be employed for commercial production.

1.-10. (canceled)

11. An improved process for the in situ preparation of Olanzapine Form I, said process comprising:
(i) dissolving substantially DMSO-free crude olanzapine in a chlorinated solvent to form a solution including olanzapine;
(ii) drying the solution including olanzapine to form a substantially water-free solution including olanzapine;
(iii) refluxing the substantially water-free solution including olanzapine; and
(iv) concentrating the substantially water-free solution including olanzapine at reduced pressure for sufficient time to precipitate olanzapine Form I crystals.

12. The improved process of claim 11, wherein a process of preparing crude olanzapine comprises:
(a) refluxing a mixture of 4-amino-2-methyl-10H-thieno[2,3-b][1,5]benzodiazepine hydrochloride, N-methyl piperazine, dimethylsulfoxide and toluene in an inert atmosphere at elevated temperature for a sufficient time.

13. The improved process of claim 11, wherein a process of preparing substantially DMSO-free crude olanzapine comprises:
(I) mixing a clean aliquot of water and crude olanzapine at a temperature in a range from 50° C. to 90° C. for a sufficient time;
(II) separating the crude olanzapine from the aqueous mixture; and
(III) repeating (a) and (b) n times, wherein n is from 1 to 4, to substantially remove DMSO and yield substantially DMSO-free crude olanzapine.

14. The improved process of claim 13, wherein n is 3 or 4.

15. The improved process of claim 11, wherein a process of drying the solution including olanzapine comprises:
(A) removing water from the solution including olanzapine using a drying agent to form a substantially water-free mixture including olanzapine;
(B) purging dry ammonia gas in the substantially water-free mixture including olanzapine; and
(C) removing the drying agent from the substantially water-free mixture including olanzapine to form a substantially water-free solution including olanzapine.

16. The improved process of claim 15, wherein the drying agent comprises an anhydrous sulfate salt selected from the group consisting of sodium sulfate and magnesium sulfate.

17. The improved process of claim 11, wherein the substantially water-free solution including olanzapine comprises water in a range from 0.0% to 0.1%.

18. The improved process of claim 11, wherein the reduced pressure is in a range from 0.789 atm to 0.855 atm.

19. The improved process of claim 11, wherein the chlorinated solvent comprises methylene chloride.

20. The improved process of claim 11, further comprising:
(v) separating the olanzapine Form I crystals from the substantially water-free solution; and
(vi) drying the olanzapine Form I crystals at reduced pressure for a sufficient time.

21. The improved process of claim 20, wherein the reduced pressure is in a range from 0.789 atm to 0.855 atm.

22. The improved process of claim 20, wherein the olanzapine Form I crystals are dried at temperature in a range from 60° C. to 70° C.

23. The improved process of claim 20, wherein the sufficient time for drying the olanzapine Form I crystals is in a range from 12 hours to 72 hours.

24. The improved process of claim 20, further comprising rinsing the olanzapine Form I crystals with a chlorinated solvent prior to drying said crystals.

25. The improved process of claim 24, wherein the chlorinated solvent comprises methylene chloride.
